(19) United States
(12) Patent Application Publication
(10) Pub. No.: US 2007/0274998 A1
(43) Pub. Date: Nov. 29, 2007

(54) Novel Bispecific Molecules For Use In Therapy And Diagnosis

(75) Inventor: Nalan Utku, Berlin (DE)

Correspondence Address: John P White;Cooper & Dunham, 1185 Avenue of the Americas, New York, NY, 10036, US

(73) Assignee: GENPATZZ PHARMACOGENTETICS AG, BERLIN GERMANY (DE)

(21) Appl. No.: 10/512,960

(22) PCT Filed: Apr. 29, 2003
(86) PCT No.: PCT/EP03/04461

§ 371 Date: Jun. 23, 2005

(30) Foreign Application Priority Data
Apr. 29, 2002 (EP) 02009292.0

(51) Int. Cl. A61K 39/395; A61P 3/10; A61P 31/00; A61P 35/00; A61P 37/00; C07H 21/00; C07K 16/28; C12N 1/00; C12N 5/04; C12N 5/08; C12P 21/00

(52) U.S. Cl. 424/173.1; 435/243; 435/325; 435/419; 530/387.1; 536/23.53

(57) ABSTRACT

Provided are bispecific molecules that are characterized by having at least a first binding domain which binds T-cell immune response cDNA 7 (TIRC7) and a second binding domain which binds T cell receptor (TCR), in particular TCR beta or gamma chain. Furthermore, compositions comprising said bispecific molecules and their use in methods of diagnosis and treating immune response related diseases are described.

The present invention relates to bispecific molecules that are characterized by having at least a first binding domain which binds T-cell immune response cDNA 7 (TIRC7) and a second binding domain which binds T cell receptor (TCR); and optionally comprising further functional domains. Furthermore, the present invention relates to compositions comprising said bispecific molecules and their use in methods of diagnosis and treating immune response related and other diseases including tumors.

Several documents are cited throughout the text of this specification. Each of the documents cited herein (including any manufacturer's specifications, instructions, etc.) are hereby incorporated herein by reference; however, there is no admission that any document cited is indeed prior art as to the present invention.

T-cell activation is a serial process involving multiple signaling pathways and sequential changes in gene expression resulting in differentiation of T-cells into distinct subpopulations, i.e. Th1 and Th2, which are distinguishable by their pattern of cytokine production and characterize the mode of cellular immune response. The T-cell response is initiated by the interaction of the antigen-specific T-cell receptor (TCR) with a peptide presented by major histocompatibility complex (MHC) molecules on the surface of antigen presenting cells (APCs). Additional signals are provided by a network of receptor-ligand interactions mediated by a number of membrane proteins such as CD28/CTLA4 and B7, CD40/CD40L, LFA-1 and ICAM-1 (Lenschow, Science 257 (1992), 789-792; Linsley, Annu. Rev. Immunol. 11 (1993), 191-212; Xu, Immunity 1 (1994), 423-431; Bachmann, Immunity 7 (1997), 549-557; Schwartz, Cell 71 (1992), 1065-1068) collectively called costimulatory signals (Perez, Immunity 6 (1997), 411). These membrane proteins can alter T-cell activation in distinct ways (Bachmann, Immunity 7 (1997), 549-557) and regulate the immune response by the integration of positive and negative signals provided by these molecules (Bluestone, Immunity 2 (1995), 555-559; Perez, Immunity 6 (1997), 411). Many of the agents which are effective in modulating the cellular immune response either interfere with the T-cell receptor (Cosimi, Transplantation 32 (1981), 535-539) block costimulatory signaling (Larsen, Nature 381 (1996), 434-438; Blazar J. Immuno. 157 (1996), 3250-3259; Kirk, Proc. Natl. Acad. Sci. USA 94 (1997), 8789-8794; Linsley, Science 257 (1992), 792-95; Turka, Proc. Natl. Acad. Sci. USA 89 (1992), 11102-11105) or inhibit intracellular activation signals downstream from these primary cell membrane triggers (Schreiber and Crabtree, Immunology Today 13 (1992), 136-42). Therapeutic prevention of T-cell activation in organ transplantation and autoimmune diseases presently relies on panimmunosupressive drugs interfering with downstream intracellular events. Specific modulation of the immune response remains a long-standing goal in immunological research. Furthermore, recent advances in understanding fundamental mechanisms of regulation of the immune response are throwing light on mechanisms of tumor growth. The understanding of the immunological aspects of tumor expansion is leading to the development of new strategies to stimulate the immune system to mount more effective responses to tumors; see, e.g., Boura et al., Hepatogastroenterology 48 (2001), 1040-1044.

In view of the need of therapeutic means for the treatment of diseases related to immune responses of the human body, the technical problem of the present invention is to provide means and methods for modulation of the immune response in a subject. The solution to said technical problem is achieved by providing the embodiments characterized in the claims, and described further below.

Accordingly, the present invention relates to a bispecific molecule that comprises a first binding domain which binds T-cell immune response cDNA 7 (TIRC7) and a second binding domain which binds T cell receptor (TCR).

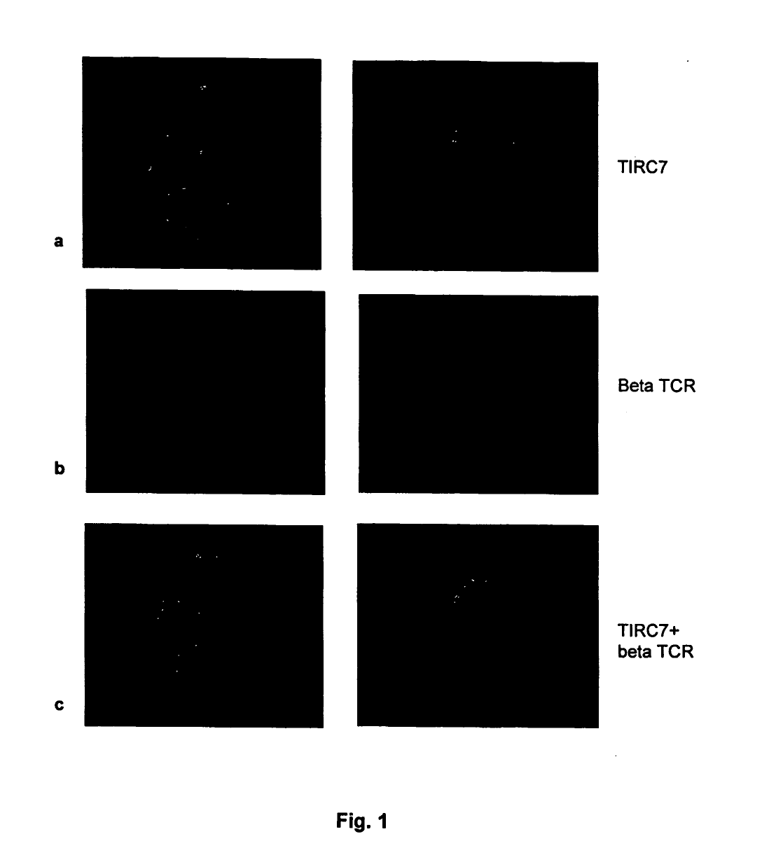

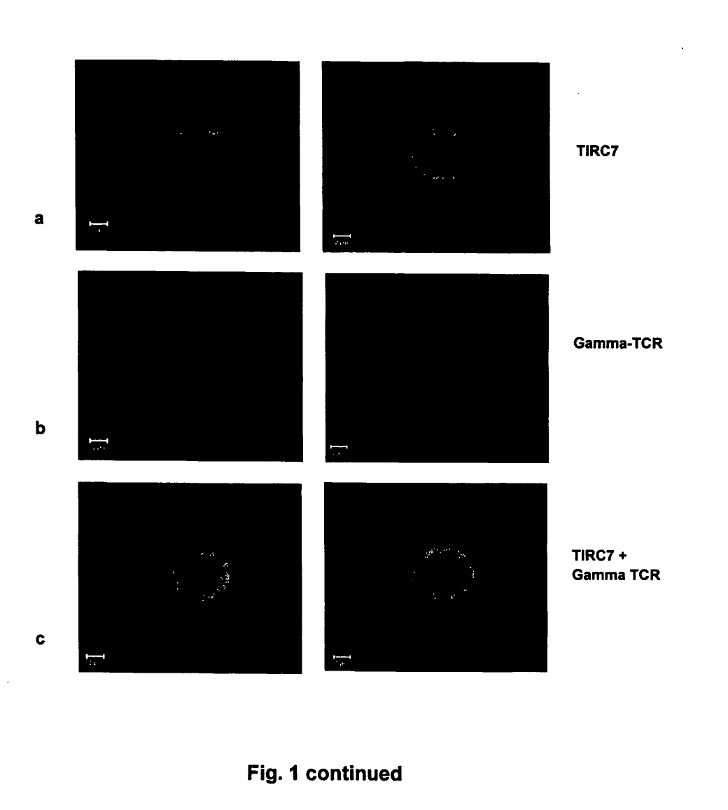

In accordance with the present invention, it was surprisingly found that T-cell immune response cDNA 7 (TIRC7) co-localizes on T cells with T cell receptor (TCR), in particular with gamma-TCR and beta-TCR; see FIG. 1. Since both proteins play a major role in immune responses and have been found by the inventors to be expressed on a specific subset of cells, it is reasonable to assume that agents modulating their interaction and/or activity will have beneficial, additive and preferably synergistic effects on the treatment of diseases and conditions, wherein TIRC7 and/or TCRs are involved in. Furthermore, such agents are expected to be useful in diagnosis, where the presence or absence of either or both proteins is associated with said disease or condition. Accordingly, the present invention provides novel bispecific molecules which have binding specificity for TIRC7 and TCR. Certain bispecific molecules of the present invention are used for binding to antigen or to block interaction of a protein and its ligand; their use to promote interactions between immune cells and target cells is however preferred. Finally, antigen-binding molecules of the invention are used to localize immune cells, tumor cells such as from leukemias and B-cell lymphomas, anti-tumor agents, target moieties, reporter molecules or detectable signal producing agents to an antigen of interest.

T cell receptors (TCRs) are well described in the art; see also supra. The receptors on T cells consist of immunoglobulin-like integral membrane glycoproteins containing 2 polypeptide subunits, alpha and beta, of similar molecular weight, 40 to 55 kD in humans. Like the immunoglobulins (Ig) of the B cells, each T-cell receptor subunit has, external to the cell membrane, an N-terminal variable (V) domain and a C-terminal constant (C) domain. The gene cluster for the beta subunit of T-cell antigen receptor is on chromosome 7 in man and on chromosome 6, near the immunoglobulin kappa light chain, in the mouse, an example of nonhomology of synteny; see, e.g., Caccia et al., Cell 37 (1984), 1091-1099; Lee et al., J. Exp. Med. 160 (1984), 905-913; Robinson et al., Proc. Nat. Acad. Sci. 90 (1993), 2433-2437; Rowen et al., Science 272 (1996), 1755-1762. Beta-TCR is thought to be involved in, for example, T-cell leukemias, T-cell lymphomas and autoimmune diseases such multiple sclerosis.

During the search for the T-cell receptor genes, Saito et al. (Saito et al., Nature 309 (1984), 757-762, Nature 312 (1984), 36-40) identified in T cells another Ig-like gene they called gamma. The product of the rearranged gamma locus is the gamma chain, which is expressed, along with the delta chain, on the surface of a subset of T lymphocytes. The gamma chain was identified as part of a heterodimer gamma-delta, associated with CD3, on the surface of CD3+/CD4−/CD8− peripheral T lymphocytes and thymocytes. The human T-cell receptor gamma (TCRG) locus was mapped to chromosome 7 and in mouse it was assigned to chromosome 13. Lefranc et al. (Lefranc et al., Cell 45 (1986), 237-246; Lefranc et al., Proc. Nat. Acad. Sci. 83 (1986), 9596-9600; Lefranc et al., Nature 319 (1986), 420-422; Lefranc and Rabbitts, Res. Immun. 141 (1990), 565-577. Trends Biochem. Sci. 14 (1989), 214-218) showed that the C-gamma-1 gene has 3 exons, whereas the C-gamma-2 gene has 4 exons including a duplicated second exon; see also Allison et al., Nature 411 (2001), 820-824. The role of gamma/delta T cells in antimicrobial immunity is firmly established; see, e.g., Kaufmann et al., Proc. Nat. Acad. Sci. 93 (1996), 2272-2279.

As mentioned before, said TCR bound by the binding domain of the bispecific molecule of the invention is gamma-TCR or beta-TCR. Further information on the genes and proteins of T cell receptors (TCRs) which can be employed in accordance with the present invention can be found in databases such as the “Human Gene Nomenclature Database”; see Guidelines for Human Gene Nomenclature, Genomics 79 (2002), 464-470.

The term “TIRC7”, also known as T-cell immune regulator 1 (TCIRG1), as used in accordance with the present invention, denotes a protein involved in the signal transduction of T-cell activation and/or proliferation and that, preferably in a soluble form is capable of inhibiting or suppressing T-cell proliferation in response to alloactivation in a mixed lymphocyte culture or in response to mitogens when exogeneously added to the culture. In vitro translated TIRC7 protein is able to efficiently suppress in a dose dependent manner the proliferation of T-cells in response to alloactivation in a mixed lymphocyte culture or in response to mitogens. TIRC7 is known to the person skilled in the art and described, inter alia, in WO99/11782; Utku et al., Immunity 9 (1998), 509-518 and Heinemann et al., Genomics 57 (1999), 398-406. Preferably, the major extracellular domain of TIRC7 (see FIG. 1 of WO99/11782) or peptides derived thereof are bound by the TIRC7 specific binding domain of the bispecific molecule of the present invention.

The TIRC7 and TCR antigen-binding sites can be obtained by any means, for example from a monoclonal antibody, or from a library of random combinations of and V_{L} and V_{H} domains.

The term “bispecific molecule” includes molecules which have at least the two mentioned binding domains directly or indirectly linked by physical or chemical means. Furthermore, the bispecific molecule of the present invention can have at least two binding domains binding TCR, i.e. the TCR beta and gamma chain, respectively. However, the bispecific molecule of the present invention may comprise in addition further functional domains such as additional binding domains and/or moieties such as a cytotoxic agent or a label and the like. Means and methods for the preparation of multivalent, multispecific molecules having at least one specificity for a desired antigen are known to the person skilled in the art. As used herein, unless otherwise indicated or clear from the context, antibody or binding domains, regions and fragments are accorded standard definitions as are well known in the art; see, e.g., Abbas et al., Cellular and Molecular Immunology (1991), W. B. Saunders Company, Philadelphia, Pa.

Bispecific molecules of the invention can cross-link antigens on target cells with antigens on immune system effector cells. This can be useful, for example, for promoting immune responses directed against cells which have a particular antigens of interest on the cell surface. According to the invention, immune system effector cells include antigen specific cells such as T cells which activate cellular immune responses and nonspecific cells such as macrophages, neutrophils and natural killer (NK) cells which mediate cellular immune responses. Hence, bispecific molecules of the invention can have a further binding site for any cell surface antigen of an immune system effector cell. Such cell surface antigens include, for example, cytokine and lymphokine receptors, Fc receptors, CD3, CD16, CD28, CD32, CD64, CD80 and CD86 (also known as B7-1 and B7-2). In general, antigen binding sites are provided by scFvs which are derived from antibodies to the aforementioned antigens and which are well known in the art. Antigen-binding sites of the invention which are specific for cytokine and lymphokine receptors can also be sequences of amino acids which correspond to all or part of the natural ligand for the receptor. For example, where the cell-surface antigen is an IL-2 receptor, an antigen-binding protein of the invention can have an antigen-binding site which comprises a sequence of amino acids corresponding or IL-2. Other cytokines and lymphokines include, for example, interleukins such as interleukin-4 (IL-4) and interleukin-5 (IL-5), and colony-stimulating factors (CSFs) such as granulocyte-macrophage CSF (GM-CSF), and granulocyte CSF (G-CSF).

In addition, any one of the described bispecific molecules may contain a binding domain binding FcgammaRI on activated effector cells. The clinical potential of this approach for the treatment of tumors such as B cell malignancies looks most attractive. Triggering of antitumor immunity by expression of anti-FcgammaR scFv on cancer cell surface has been described by Gruel et al., Gene Ther. 8 (2001), 1721-1728. In addition or alternatively, the bispecific molecule of the invention may comprise a binding domain binding CD3. This embodiment is particularly useful for the treatment of carcinoma; see, e.g., Riesenberg et al., J. Histochem. Cytochem. 49 (2001), 911-917, which report on the lysis of prostate carcinoma cells by trifunctional bispecific antibodies (alpha EpCAM×alpha CD3).

In a preferred embodiment, the bispecific molecule of the invention comprises at least one further binding domain binding HLA-(Human Leukocyte associated Antigens), preferably HLA class II alpha 2 chain. HLA class II antibodies which may be used in accordance with the present invention are described in Valerius et al., Leuk. Lymphoma 26 (1997), 261-269 and are also available from commercial firms; see infra. Furthermore, WO99/59633 describes multimeric molecules with at least one specificity for the HLA class II invariant chain (Ii) and their use for the clearance of therapeutic or diagnostic agents, autoantibodies, anti-graft antibodies, and other undesirable compounds.

These and other combinations of functional domains in the bispecific molecule of the present invention and uses thereof are encompassed by the present invention.

General strategies for preparation of multispecific molecules are known in the art; see; e.g., Tomlinson et al., Methods Enzymol. 326 (2000), 461-479. For example, intermediate molecular weight recombinant bispecific and trispecific antibodies by efficient heterodimerization of single chain variable domains through fusion to a Fab-chain are described in Schoonjans et al., Biomol. Eng. 17 (2001), 193-202. Dimeric and trimeric antibodies with high avidity for cancer targeting are described in Kortt et al., Biomol. Eng. 18 (2001), 95-108. Trispecific antibodies directed against CD2, CD3, and CD28 and stimulating rheumatoid arthritis T cells to produce Th1 cytokines have been described in Wong et al., Scand. J. Rheumatol. 29 (2000), 282-287. All the means, methods and applications described in the mentioned publications can be applied and adapted to the bispecific molecule of the present invention and used in accordance with teaching disclosed herein.

Once a bispecific molecule has been produced in accordance with the present invention, various assays are available to demonstrate dual or multivalent specificity of the bispecific molecules of the invention such as direct and quantitative binding assays; see, e.g., WO94/13804, WO01/80883, WO01/90192 and the mentioned publications. Biologically active bispecific molecules, for example those supposed to have anti-tumor effect can be tested in well known in vitro test set-ups and also in mouse-tumor models; see review in Beun et al., Immunol. Today 21 (1994), 2413.

Preferably, the bispecific molecule of the present invention is a bispecific immunoglobulin, wherein the first binding domain is a first immunoglobulin variable region, and the second binding domain is a second immunoglobulin variable region recognizing TIRC7 and TCR, respectively. Such immunoglobulin variable regions can be obtained from polyclonal or monoclonal antibodies as well as from phage display and other screening techniques for immunoglobulin like binding proteins. As mentioned, antibodies can be monoclonal antibodies, polyclonal antibodies but also synthetic antibodies as well as fragments of antibodies, such as Fab, Fv or scFv fragments etc. Antibodies or fragments thereof can be obtained by using methods which are described, e.g., in Harlow and Lane “Antibodies, A Laboratory Manual”, CSH Press, Cold Spring Harbor, 1988 or EP-A 0 451 216 and references cited therein. For example, surface plasmon resonance as employed in the BIAcore system can be used to increase the efficiency of phage antibodies which bind to an epitope of TIRC7 or TCR (Schier, Human Antibodies Hybridomas 7 (1996), 97-105; Malmborg, J. Immunol. Methods 183 (1995), 7-13). The production of chimeric antibodies is described, for example, in WO89/09622. Methods for the production of humanized antibodies are described in, e.g., EP-A1 0 239 400 and WO90/07861. A further source of antibodies to be utilized in accordance with the present invention are so-called xenogeneic antibodies. The general principle for the production of xenogeneic antibodies such as human antibodies in mice is described in, e.g., WO 91/10741, WO 94/02602, WO 96/34096 and WO 96/33735.

Polyclonal and monoclonal antibodies against TIRC7 are described in WO99/11782 and Utku et al., Immunity 9 (1998), 509-518. Particularly useful antibodies as a source for TIRC7 binding domains for the generation of a bispecific molecule of the invention are described in European patent application EP 0113 0730.3 filed on Dec. 21, 2001 and followed up in its subsequent PCT application.

Antibodies against TCR such as those specific for gamma-TCR and beta-TCR can be purchased from commercial firms offering immunochemical reagents, for example from Abcam Ltd, Cambridge, UK; Ortho Diagnostic Systems, Raritan, N.J.; Becton Dickenson Immunological Reagents, Mountain View, Calif.; Coulter Diagnostics, Hialeach, Fla.; Sigma Chemical Co., St. Louis, Mo.; Boehringer Mannheim, Indianapolis, Ind.; Olympus Corp., Lake Success, N.Y. All these MAbs were developed by different groups. These firms offer MAbs not only as purified, plain IgG, but also in fluorescein-conjugated forms. Furthermore, bispecific F(ab′)2 antibodies to mimic TCR/co-receptor engagement during thymocyte differentiation, which may be used in accordance with the present invention are described in Bommhardt et al., Eur. J. Immunol. 27 (1997), 1152-1163.

As mentioned before, the bispecific molecule of the present invention can be a dimeric, multimeric or a single chain molecule. In single chain bispecific molecules the binding domains, preferably Fv regions, are linked by a peptide linker, which allows the domains to associate to form a functional antigen binding site; see, e.g., WO88/09344, WO92/01047. Peptide linkers used to produce scFvs are flexible peptides selected to assure proper three-dimensional folding and association of the V_{L} and V_{H} domains and maintenance of target molecule binding-specificity. Generally, the carboxy terminus of the V_{L} or V_{H} sequence is covalently linked by such a peptide linker to the amino terminus of a complementary V_{H} or V_{L} sequence. The linker is generally 10 to 50 amino acid residues, but any length of sufficient flexibility to allow formation of the antigen binding site is contemplated. Preferably, the linker is 10 to 30 amino acid residues. More preferably the linker is 12 to 30 amino acid residues. Most preferably is a linker of 15 to 25 amino acid residues. Example of such linker peptides include three times (Gly-Gly-Gly-Gly-Ser).

In a preferred embodiment, the bispecific molecule of the present invention is a bispecific antibody. The bispecific antibodies may comprise Fc constant regions, for example for association of the polypeptide chains comprising the binding domains. In addition to providing for association of the polypeptide chains, Fc constant domains contribute other immunoglobulin functions. The functions include activation of complement mediated cytotoxicity, activation of antibody dependent cell-mediated cytotoxicity and Fc receptor binding. When antigen-binding proteins of the invention are administered for treatment or diagnostic purposes, the Fc constant domains can also contribute to serum halflife. The Fc constant domains can be from any mammalian or avian species. When antigen binding proteins of the invention are used for treatment of humans, constant domains of human origin are preferred, although the variable domains can be non-human. In cases where human variable domains are preferred, chimeric scFvs can be used. Further means and methods for the production of bispecific antibodies are described in the art; see, e.g., WO97/14719 which describes a process for producing bispecific or bivalent double head antibody fragments, which are composed of a binding complex containing two polypeptide chains, and WO01/80883. Furthermore, the bispecific molecules of the invention can be optimized in their avidity for antigen(s) while maintaining their ability to function as a natural antibody, including the ability to activate complement mediated cytotoxicity and antibody dependent cellular toxicity; see, e.g., WO01/90192.

The bispecific molecules of the present invention preferably have a specificity at least substantially identical to the binding specificity of the natural ligand or binding partner of the TIRC7 or TCR protein, in particular if TIRC7 stimulation is desired. A binding domain binding TIRC7 or TCR can have a binding affinity of at least 10⁻⁵ M, preferably higher than 10⁻⁷ M and advantageously up to 10⁻¹⁰ M. In a preferred embodiment, the bispecific molecule has an affinity of at least about 10⁻⁷ M, preferably at least about 10⁻⁹ M and most preferably at least about 10⁻¹¹ M for either or both TIRC7 and TCR. In another embodiment the bispecific molecule has an affinity of less than about 10⁻⁷ M, preferably less than about 10⁻⁶ M and most preferably in order of 10⁻⁵ M for either or both TIRC7 and TCR.

Furthermore, the present invention relates to a nucleic acid molecule or a composition of nucleic molecules encoding the bispecific molecule of the present invention. In particular, said nucleic acid molecules encode at least the binding domains, for example the variable region of an immunoglobulin chain of any one of the before described antibodies. The nucleic acid molecules are preferably operably linked to expression control sequences. Usually, the nucleic acid molecule(s) will be part of (a) vector(s), preferably expression vectors used conventionally in genetic engineering, for example, plasmids; see also the references cited herein. In addition, the present invention relates to a cell comprising the nucleic acid molecule or composition described above. The cell may be a prokaryotic host cell including gram negative as well as gram positive bacteria such as, for example, E. coli, S. typhimurium, Serratia marcescens and Bacillus subtilis, or a eukaryotic cell or cell line including yeast, higher plant, insect and preferably mammalian cells, most preferably NSO and CHO cells. Preferably, said cell is capable of expressing the bispecific molecule of the invention, for example such that the bispecific molecule or its subunits are secreted through the cell membrane. Suitable source cells for the DNA sequences and host cells for immunoglobulin expression and secretion can be obtained from a number of sources, such as the American Type Culture Collection (“Catalogue of Cell Lines and Hybridomas,” Fifth edition (1985) Rockville, Md., U.S.A., which is incorporated herein by reference). The present invention also envisages cells, which express the bispecific molecule of the invention or its binding domains such that they are localized on the cell membrane. In this embodiment, the bispecific molecule of the invention or its binding domains may function as cell membrane receptors, for example for the attraction of complement cells.

The present invention also relates to a method for producing the bispecific molecule of the invention comprising cross-linking a first binding domain which binds TIRC7 and a second binding domain which binds TCR. Conventional techniques for the production of bispecific proteins, preferably antibody fragments, are known to person skilled in the art; see, e.g., WO98/04592 and references cited therein. Starting material such as intact antibodies can be obtained according to methods known in the prior art; see literature cited supra and Current Protocols in Immunology, J. E. Codigan, A. M. Krvisbeck, D. H. Margulies, E. M. Shevack, W. Strober eds., John Wiley+Sons. It is also known from the art how to carry out the individual reaction and purification steps; see the example and, e.g., Brennan et al. Science 229 (1985), 81-83; Jung et al. Eur. J. Immunol. 21 (1991), 2491-2495.

The present invention also relates to a method for producing a bispecific molecule of the present invention comprising culturing the above described cell under appropriate conditions and isolating the bispecific molecule or portions thereof. A variety of chemical and recombinant methods have been developed for the production of bispecific and/or multivalent molecules such as antibody fragments. For review, see Holliger and Winter, Curr. Opin. Biotechnol. 4 (1993), 446-449; Carter et al., J. Hematotherapy 4 (1995), 463-470; Plückthun and Pack, Immunotechnology 3 (1997), 83-105. For example, bispecificity and/or bivalency has been accomplished by fusing two scFv molecules via flexible linkers, leucine zipper motifs, CHCL-heterodimerization, and by association of scFv molecules to form bivalent mono-specific diabodies and related structures. Multispecificity or multivalency has been achieved by the addition of multimerization sequences at the carboxy or amino terminus of the scFv or Fab fragments, by using for example, p53, streptavidin and helix-turnhelix motifs. For example, by dimerization via the helix-turn-helix motif of an scFv fusion protein of the form (scFv1)-hinge-helix-turn-helix-(scFv2), a tetravalent bispecific miniantibody is produced having two scFv binding sites for each of two target antigens. Production of IgG type bispecific antibodies, which resemble IgG antibodies in that they possess a more or less complete IgG constant domain structure, has been achieved by chemical cross-linking of two different IgG molecules or by co-expression of two antibodies from the same cell. Chemical cross-linking is described in, e.g., Merchant et al., Nat. Biotechnology 16 (1998), 677-681. Furthermore, the production of homogeneous population of bivalent, bispecific molecules that bind to one antigen at one end and to a second antigen at the other end are described; see, e.g., Colonna and Morrison, Nat. Biotechnology 15 (1997), 159-163. Further means and methods for the expression and purification of bispecific molecules such as bispecific recombinant antibody fragments derived from antibodies are known in the art; see, e.g., Dincq et. al, Protein Expr. Purif. 22 (2001), 11-24.

Furthermore, the present invention relates to a composition comprising in one or more compartments, the bispecific molecule or chemical derivatives thereof, the nucleic acid molecule or above described composition or the cell of the invention. The composition of the present invention may further comprise a pharmaceutically acceptable carrier. The term “chemical derivative” describes a molecule that contains additional chemical moieties that are not normally a part of the base molecule. Such moieties may improve the solubility, half-life, absorption, etc. of the base molecule. Alternatively the moieties may attenuate undesirable side effects of the base molecule or decrease the toxicity of the base molecule. Examples of such moieties are described in a variety of texts, such as Remington's Pharmaceutical Sciences. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intra-muscular, topical or intradermal administration. Aerosol formulations such as nasal spray formulations include purified aqueous or other solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier.

The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently. A typical dose can be, for example, in the range of 0.001 to 1000 μg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. Dosages will vary but a preferred dosage for intravenous administration of DNA is from approximately 10⁶ to 10¹² copies of the DNA molecule. The compositions of the invention may be administered locally or systemically. Administration will generally be parenterally, e.g., intravenously; DNA may also be administered directly to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as interleukins or interferons depending on the intended use of the pharmaceutical composition.

In a preferred embodiment, the pharmaceutical composition of the present invention comprises at least one further therapeutically effective agent, preferably an immunosuppressive drug, e.g., ATG, ALG, OKT3, Azathioprine, Mycophenylate, Mofetyl, Cyclosporin A, FK506, Sirolimus (Rapamune) and/or corticosteroids. Furthermore, the pharmaceutical composition may also be formulated as a vaccine, for example, if the pharmaceutical composition of the invention comprises a bispecific molecule described above for passive immunization. In addition, the bispecific molecules of the present invention can be used as in vivo immune enhancers similar as the conjugates described in U.S. Pat. No. 6,197,298. Thus, the bispecific molecules of the present invention are expected to be useful for modulating the immune system by inducing or suppressing specifically the polyclonal activation, proliferation, and/or lymphokine production of T lymphocytes, or subsets thereof. Potentiation of the immune system is desirable for treating a number of pathological conditions, e.g., for treatment of malignant tumors, such as those associated with renal cell carcinoma, malignant melanoma, colon carcinoma, and small cell lung carcinoma or for the treatment of infectious diseases, or to protect individuals exposed to infectious agents from contracting the infections. Infectious diseases appropriate for treatment with immune potentiators include hepatitis, and particularly hepatitis B and C, herpes simplex I and II, condyloma, influenza, and pneumonia. Immune potentiators may also be used as adjuvants for vaccines, which could reduce the number of times that a vaccine needs to be administered in order to be effective in prophylaxis. This could be particularly effective for vaccination against diphtheria, influenza, and measles, as there already are mass vaccination programs for children against these diseases. The bispecific molecules of the present invention could also be used in veterinary practice, particularly to treat companion animals affected with cancers or chronic infections. For use in veterinary practice, the same substances of the invention mentioned above are employed, with the fragments and antibodies targeting the T cell antigen of the animal one is seeking to treat. Among the diseases in companion animals which might be particularly well suited for treatment with the products of the invention are the canine distemper adenovirus, corona-virus, or Rabies virus, and the feline leukemia virus.

Therapeutic or diagnostic compositions of the invention are administered to an individual in a therapeutically effective dose sufficient to treat or diagnose disorders as mentioned above. The effective amount may vary according to a variety of factors such as the individual's condition, weight, sex and age. Other factors include the mode of administration. In addition, co-administration or sequential administration of other agents may be desirable. A therapeutically effective dose refers to that amount of bispecific molecule of the invention sufficient to ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

For use in diagnosis, a variety of techniques are available for labeling biomolecules, are well known to the person skilled in the art and are considered to be within the scope of the present invention. Such techniques are, e.g., described in Tijssen, “Practice and theory of enzyme immuno assays”, Burden, R H and von Knippenburg (Eds), Volume 15 (1985), “Basic methods in molecular biology”; Davis L G, Dibmer M D; Battey Elsevier (1990), Mayer et al., (Eds) “Immunochemical methods in cell and molecular biology” Academic Press, London (1987), or in the series “Methods in Enzymology”, Academic Press, Inc. There are many different labels and methods of labeling known to those of ordinary skill in the art. Commonly used labels comprise, inter alia, fluorochromes (like fluorescein, rhodamine, Texas Red, etc.), enzymes (like horse radish peroxidase, β-galactosidase, alkaline phosphatase), radioactive isotopes (like ³²P or ¹²⁵I), biotin, digoxygenin, colloidal metals, chemi- or bio-luminescent compounds (like dioxetanes, luminol or acridiniums). Labeling procedures, like covalent coupling of enzymes or biotinyl groups, iodinations, phosphorylations, biotinylations, random priming, nick-translations, tailing (using terminal transferases) are well known in the art. Detection methods comprise, but are not limited to, autoradiography, fluorescence microscopy, direct and indirect enzymatic reactions, etc. In addition, the above-described compounds etc. may be attached to a solid phase. Solid phases are known to those in the art and may comprise polystyrene beads, latex beads, magnetic beads, colloid metal particles, glass and/or silicon chips and surfaces, nitrocellulose strips, membranes, sheets, animal red blood cells, or red blood cell ghosts, duracytes and the walls of wells of a reaction tray, plastic tubes or other test tubes. Suitable methods of immobilizing bispecific molecules of the invention on solid phases include but are not limited to ionic, hydrophobic, covalent interactions and the like. The solid phase can retain one or more additional receptor(s) which has/have the ability to attract and immobilize the region as defined above. This receptor can comprise a charged substance that is oppositely charged with respect to the reagent itself or to a charged substance conjugated to the capture reagent or the receptor can be any specific binding partner which is immobilized upon (attached to) the solid phase and which is able to immobilize the reagent as defined above.

Commonly used detection assays can comprise radioisotopic or non-radioisotopic methods. These comprise, inter alia, RIA (Radioisotopic Assay) and IRMA (Immune Radioimmunometric Assay), EIA (Enzyme Immuno Assay), ELISA (Enzyme Linked Immuno Assay), FIA (Fluorescent Immuno Assay), and CLIA (Chemiluminescent Immune Assay). Other detection methods that are used in the art are those that do not utilize tracer molecules. One prototype of these methods is the agglutination assay, based on the property of a given molecule to bridge at least two particles.

The present invention also relates to a kit comprising a bispecific molecule of the invention. Such kits are useful for a variety of purposes including but not limited to forensic analyses, diagnostic applications, and epidemiological studies in accordance with the above-described diseases and disorders. Such a kit would typically comprise a compartmentalized carrier suitable to hold in close confinement at least one container. The carrier would further comprise reagents for detection such as labeled antigen or enzyme substrates or the like.

As described before, the composition of the present invention is useful in diagnosis, prophylaxis, vaccination or therapy. Accordingly, the present invention relates to the use of the bispecific molecule, the nucleic acid molecule or composition or the cell of the present invention for the preparation of a pharmaceutical or diagnostic composition for the treatment of diseases related to a disorder of the immune response, preferably for the treatment of graft versus host disease, autoimmune diseases, multiple sclerosis, lupus erythematosus, allergic diseases, infectious diseases, sepsis, diabetes, for the treatment of tumors, for the improvement of wound healing or for inducing or maintaining immune unresponsiveness in a subject. Preferably, the tumor to be treated or diagnosed is selected from the group consisting of prostate cancer, breast cancer, glioblastoma, medulloblastoma, astrocytoma, primitive neuroectoderma, brain stem glioma cancers, colon carcinoma, bronchial carcinoma, squamous carcinoma, sarcoma, carcinoma in the head/neck, T cell lymphoma, B cell lymphoma, mesothelioma, leukemia and meningeoma.

For these embodiments, the bispecific molecules of the invention can be chemically or bio-synthetically linked to anti-tumor agents or detectable signal-producing agents; see also supra. Antitumor agents linked to a bispecific molecule, for example a bispecific antibody, include any agents which destroy or damage a tumor to which the antibody has bound or in the environment of the cell to which the antibody has bound. For example, an anti-tumor agent is a toxic agent such as a chemotherapeutic agent or a radioisotope. Suitable chemotherapeutic agents are known to those skilled in the art and include anthracyclines (e.g. daunomycin and doxorubicin), methotrexate, vindesine, neocarzinostatin, cis-platinum, chlorambucil, cytosine arabinoside, 5-fluorouridine, melphalan, ricin and calicheamicin. The chemotherapeutic agents are conjugated to the antibody using conventional methods; see, e.g., Hermentin and Seiler, Behring Inst. Mitt. 82 (1988), 197-215.

Detectable signal-producing agents are useful in vivo and in vitro for diagnostic purposes. The signal producing agent produces a measurable signal which is detectable by external means, usually the measurement of electromagnetic radiation. For the most part, the signal producing agent is an enzyme or chromophore, or emits light by fluorescence, phosphorescence or chemiluminescence. Chromophores include dyes which absorb light in the ultra-violet or visible wavelength range, and can be substrates or degradation products of enzyme catalyzed reactions.

The invention further contemplates bispecific molecules of the invention to which target or reporter moieties are linked. Target moieties are first members of binding pairs. Anti-tumor agents, for example, are conjugated to second members of such pairs and are thereby directed to the site where the antigen-binding protein is bound. A common example of such a binding pair is adivin and biotin. In a preferred embodiment, biotin is conjugated to an bispecific molecule of the invention, and thereby provides a target for an anti-tumor agent or other moiety which is conjugated to avidin or streptavidin. Alternatively, biotin or another such moiety is linked to a bispecific molecule of the invention and used as a reporter, for example in a diagnostic system where a detectable signal-producing agent is conjugated to avidin or streptavidin. Suitable radioisotopes for use as anti-tumor agents are also known to those skilled in the art. For example, ¹³¹I or ²¹¹At is used. These isotopes are attached to the antibody using conventional techniques; see, e.g., Pedley et al., Br. J. Cancer 68 (1993), 69-73. Alternatively, the anti-tumor agent which is attached to the antibody is an enzyme which activates a prodrug. In this way, a prodrug is administered which remains in its inactive form until it reaches the tumor site where it is converted to its cytotoxic form once the antibody complex is administered. In practice, the antibody-enzyme conjugate is administered to the patient and allowed to localize in the region of the tissue to be treated. The prodrug is then administered to the patient so that conversion to the cytotoxic drug occurs in the region of the tissue to be treated. Alternatively, the anti-tumor agent conjugated to the antibody is a cytokine such as interleukin-2 (IL-2), interleukin-4 (IL-4) or tumor necrosis factor alpha (TNF-α). The antibody targets the cytokine to the tumor so that the cytokine mediates damage to or destruction of the tumor without affecting other tissues. The cytokine is fused to the antibody at the DNA level using conventional recombinant DNA techniques.

The present invention further provides methods of treating a mammal having an undesirable condition associated with a disease as defined above, comprising administering to the mammal a therapeutically effective dose of any one of the above described bispecific molecules of the invention.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. arresting its development; or (c) relieving the disease, i.e. causing regression of the disease.

Compositions comprising the bispecific molecule of this invention can be added to cells in culture (in vitro) or used to treat patients, such as mammals (in vivo). Where the bispecific molecule is used to treat a patient, the bispecific molecule is preferably combined in a pharmaceutical composition with a pharmaceutically acceptable carrier such as a larger molecule to promote stability or a pharmaceutically acceptable buffer that serves as a carrier for the bispecific molecule that has more than one unit coupled to a single entity. The methods of the invention include administering to a patient, preferably a mammal, and more preferably a human, the composition of the invention in an amount effective to produce the desired effect. The bispecific molecule can be administered as a single dose or in multiple doses. Useful dosages of the active agents can be determined by comparing their in vitro activity and the in vivo activity in animal models. For example, methods of ex vivo immunization using heterologous intact bispecific and/or trispecific antibodies are described in EP-A-885 614 and induction of a long-lasting antitumor immunity by a trifunctional bispecific antibody is reported in Ruf and Lindhofer, Blood 98 (2001), 2526-2534.

Methods for extrapolation of effective dosages in mice, and other animals, to humans are known in the art. The present invention also provides a method of modulating (e.g., activating or inhibiting) immune cell (e.g., T-cells, B-cells, NK cells, LAK cells, or dendritic cells) activation, proliferation, and/or differentiation that includes contacting an immune cell with a bispecific molecule described above.

These and other embodiments are disclosed and encompassed by the description and examples of the present invention. Further literature concerning any one of the antibodies, methods, uses and compounds to be employed in accordance with the present invention may be retrieved from public libraries and databases, using for example electronic devices. For example the public database “Medline” may be utilized which is available on the Internet, for example under http://www.ncbi.nlm.nih.gov/PubMed/medline.html. Further databases and addresses, such as http://www.ncbi.nlm.nih.gov/, http://www.infobiogen.fr/, http://www.fmi.ch/biology/research_tools.html, http://www.tigr.org/, are known to the person skilled in the art and can also be obtained using, e.g., http://www.lycos.com. An overview of patent information in biotechnology and a survey of relevant sources of patent information useful for retrospective searching and for current awareness is given in Berks, TIBTECH 12 (1994), 352-364.

It is to be understood and expected that variations in the principles of invention herein disclosed may be made by one skilled in the art and it is intended that such modifications are to be included within the scope of the present invention.

The examples which follow further illustrate the invention, but should not be construed to limit the scope of the invention in any way. Detailed descriptions of conventional methods, such as those employed in the construction of vectors and plasmids, the insertion of genes encoding polypeptides into such vectors and plasmids, the introduction of plasmids into host cells, and the expression and determination thereof of genes and gene products can be obtained from numerous publication, including Sambrook et al., (1989) Molecular Cloning: A Laboratory Manual, 2nd ed., Cold Spring Harbor Laboratory Press. Particularly useful means and methods for the recombinant production of bispecific molecules are described in WO94/13804, WO01/80883 and WO01/90192. All references mentioned herein are incorporated in their entirety.

The FIGURE shows.

FIG. 1: FITC staining of activated T cells with anti-TIRC7 and anti-TCR (gamma-TCR or beta-TCR) antibodies. TIRC7 (a) and TCR (gamma-TCR or beta-TCR) (b) are co-localized on the cell membrane of human 48 h activated T cell as shown in (c) (TIRC7+beta-TCR and TIRC7+gamma-TCR).

The examples illustrate the invention.

### EXAMPLE 1

### Co-Localization of TIRC7 and TCR (Gamma-TCR and Beta-TCR)

Human PBMC were activated with PHA for two to three days and attached to slides for further confocal microscopic analysis as described in Utku et al, Immunity, 1998. A specific anti-TIRC7 polyclonal antibody Ab 79 was used for staining of TIRC7 protein and indirectly labeled with FITC, for TCR gamma and beta receptor mAbs (Santa Cruz) were used and indirectly labeled with PE. The result is shown in FIG. 1.

### EXAMPLE 2

### Production of Bispecific F(ab′)₂ Antibody Fragments

In principle, intact polyclonal or monoclonal anti-TIRC7 and anti-TCR antibodies, respectively, see supra, can be used to prepare bispecific antibody fragments; see, e.g., Brennan et al., Science 229 (1985), 81-83. For example, intact anti-TIRC7 and anti-TCR gamma or beta antibodies used in Example 1 are fragmented by peptic digestion (three hours at 37° C. in acetate buffer of pH 4.0, Pepsin from Sigma) to F(ab′)₂ fragments to cleave off the Fc portion of the antibody. The reaction is terminated by increasing the pH value to 8 with Tris buffer and the resulting F(ab′)2 fragments are purified by column chromatography (e.g. Superdex 200 column). Then, the disulfide bonds of the hinge region of the purified F(ab′)₂ molecule are digested by reduction in the presence of arsenite and the F(ab′)-SH fragments thus obtained are again purified by column chromatography, so as to then modify the reduced SH groups with the Ellman's reagent (DTNB) to F(ab′)-TNB (incubation for 20 hours at room temperature with an equal volume of a mixture of 5,5′-dithiobis-2-nitro-benzoic acid (DTNB; Sigma) and thionitrobenzoate (TNB) with a molar ratio of the DTNB-TNB mixture of 20:30 and adjustment by incubating for a few minutes a 40 mM DTNB solution with a 10 mM DTT solution). After further purification by column chromatography one of the two antibody fragments is reduced to F(ab′)-SH (0.1 mM DTT (Sigma) for one hour at 25° C.), purified by column chromatography and hybridized to the other F(ab′)-TNB fragment (1 hr at 25° C.) to give a bispecific F(ab′)₂ fragment. Finally, the bispecific antibody fragments thus obtained are purified by gel chromatography.

The bispecific molecule may be further modified, for example labeled with a fluorescent dye and tested, inter alia, for the binding to human tumor material, the activity in lymphocyte proliferation and cytotoxicity tests and the stability under in vivo conditions, for example incubation in human serum at 37° C.

1. A bispecific molecule that comprises a first binding domain which binds T-cell immune response cDNA 7 (TIRC7) and a second binding domain which binds T cell receptor (TCR).

2. The bispecific molecule of claim 1, wherein said TCR is beta-TCR or gamma-TCR.

3. The bispecific molecule of claim 1 which is a single chain or a dimeric or multimeric molecule.

4. The bispecific molecule of claim 1 which has at least one further functional domain.

5. The bispecific molecule of claim 1 which is a bispecific antibody.

6. A nucleic acid molecule or a composition of nucleic acid molecules encoding the bispecific molecule of claim 1.

7. The nucleic acid molecule or composition of claim 6, wherein any one of said nucleic acid molecules is operably linked to expression control sequences.

8. A cell transformed with the nucleic acid molecule or composition of claim 6.

9. A method for producing a bispecific molecule of claim 1 comprising cross-linking a first binding domain which binds TIRC7 and a second binding domain which binds T cell receptor (TCR).

10. A method for producing a bispecific molecule comprising culturing the cell of claim 8 under appropriate conditions and isolating the bispecific molecule or portions thereof.

11. A composition comprising in one or more compartments, the bispecific molecule of claim 1 and optionally a pharmaceutically acceptable carrier.

12. The composition of claim 11 for use in diagnosis, prophylaxis, vaccination or therapy.

13. The use of the bispecific molecule of claim 1 for the preparation of a pharmaceutical composition for the treatment of diseases related to a disorder of the immune response, preferably for the treatment of graft versus host disease, autoimmune diseases, allergic diseases, infectious diseases, sepsis, diabetes, for the treatment of tumors, for the improvement of wound healing or for inducing or maintaining immune unresponsiveness in a subject.

14. A method of treating a mammal having an undesirable condition associated with a disease comprising administering to the mammal a therapeutically effective dose of bispecific molecules of claim 1.
